(19) United States
(12) Patent Application Publication
(10) Pub. No.: US 2013/0144308 A1
(43) Pub. Date: Jun. 6, 2013

(54) Blackhead and Blemish Extractor

(71) Applicant: Diana Forrest, Richmond Hill (CA)

(72) Inventor: Diana Forrest, Richmond Hill (CA)

(73) Assignee: Diana Forrest, Richmond Hill (CA)

(21) Appl. No.: 13/311,861

(22) Filed: Dec. 6, 2011

(51) Int. Cl. A61B 17/50

(52) U.S. Cl. 606/131

(57) ABSTRACT

The current invention is intended to be a disinfectable blemish remover. Blemish means blackhead, whitehead, pimple or ingrown hair. Said invention is for use to remove blemishes without having a greater risk of infection. Disinfectable or disposable parts tool extracts blemishes with minimal trauma to the skin and decreased spread of bacteria.

Remover is comprised of two handles having two shaped arms with disposable or fixed polymer clamping wedges in contact with each other, pivot means coupling the handles and the arms, characterized in that the part of each handle through which the pivot extends defines an integral extension extending from the pivot outward over part of the arm to support and apply pressure onto said polymer clamps during the pinching movement to remove said blemish.

### SUMMARY OF THE INVENTION

Accordingly, the present invention is a hand-held apparatus for the removal of blemishes of the skin which comprises: opposing handle members attached to arm members which have a disposable or fixed sterile clamping mechanism on its end, pivotally attached at the hinge point. The intention for this invention is for a person to safely remove a blemish with the utmost hygiene without spreading infectious bacteria. Said polymer wedges extract the blemish quickly and efficiently and can be replaced (or disinfected) after every usage to prevent spreading of infectious bacteria.

### BACKGROUND OF THE INVENTION:

Four processes are thought to be involved in the pathogenesis of acne: sebum production, ductal hypercornification, and bacterial colonization of an epidermal pore resulting in inflammation. Clinically, acne is characterized by the appearance of comedones (non-inflamed lesions) and inflamed lesions (papules and pustules). Ductal hypercornification is caused by the increased proliferation of the basal ductal keratinocytes and increased adhesion between the corneocytes of the duct wall. This leads to a build-up of corneocytes in the duct lumen which becomes mixed with sebum produced by the sebaceous gland, resulting in follicular impactions. If the correct conditions prevail, the follicular impactions can develop into comedones.

Acne occurs most frequently in young (adolescent) adults, it is not always resolved by the end of the teenage years and may persist into the 40′s for certain individuals. In some cases, the onset of acne may not occur until the mid-20′s.

For all the above reasons, it is important to recognize that acne is a significant medical condition, and that it affects a significant part of the population, and many age groups.

Acne treatment includes diet restrictions, antibiotics (both oral and topical), as well as exposure to sunlight and other light sources. Retinoids and hormonal manipulation treatment are also used. Diet restrictions are typically problematic, with unpredictable results. Oral antibiotics have been successfully used to treatment acne, but can have disadvantages, including yeast vaginitis, gastrointestinal side effects and photosensitivity. Retinoids are not advised for women of child-bearing potential and often cause xerosis (dry skin), erythema, cheilitis, conjunctival irritation and alopecia, while hormonal therapy expose the patient to risk of thromboembolism, feminization (in men) and other undesirable effects. Topical antibiotic applications are also used for acne treatment, including retinoids, benzoyl peroxide, salicylic acid and antibiotics. Each of these has their specific undesirable side effects, including undesirable skin surface reactions in some cases.

### DESCRIPTION OF THE INVENTION

The preferred embodiment of the present invention is disclosed. In particular, the present invention is a hand-held apparatus for removal of skin blemishes and may be used by individuals who suffer from mild to moderate cases of skin acne as a way to extract blemishes with a lesser chance of further complications commonly associated with acne treatment such as incomplete treatment, infection, scarring or bruising around the treated area.

The hand-held apparatus for extraction of blackheads and/or blemishes includes opposing handle members attached to arm members with sterile clamping wedges functionally attached. Said handle members are pivotally attached to one another at a hinge point. The opposing handle members have closed (or partially closed) loops or straight arms and may include polymer grip pads such that the apparatus will not slip out of a users grasp. Additionally, the handle members have a curvature such that the ends of the handle members, where the arm members are attached, are substantially non-planar with the handle members. Additionally, extending from one of the two finger loops, the apparatus includes a thin, finger-like tab with a sharp edge. By holding the tool parallel to the skin's surface and applying lateral pressure, the extension tab presses out blemishes. This allows for removal of blemishes in areas difficult to access using the clamping end of the tool.

In another embodiment, the hand-held apparatus for removal of blackheads and blemishes will also have a light source functionally attached to allow a user a better view of the area to be treated. Said light source may be a battery powered light source such as an L.E.D. or a small mirror and may be fixed, adjustable, or removable. Often times the area to be treated will be small or in an area that is hard to see unaided, and it will be appreciated by one of ordinary skill in the art that a light source would be a beneficial addition.

This blemish extractor invention is intended to remove blemishes otherwise known as blackheads, whiteheads, pimples or ingrown hairs. The apparatus clamps the skin around a blemish and with pressure applied by bringing the opposing handle members together, the contents of the affected pore is extruded. The two handles having two shaped arms with disposable or fixed polymer clamping wedges in contact with each other, pivot means coupling the handles and the arms, characterized in that the part of each handle through which the pivot extends defines an integral extension extending from the pivot outward over part of the arm to support and apply pressure onto said polymer clamps during the pinching movement to remove said blemish. The tips of the invention are small enough to grasp the immediate area surrounding a blemish. The two arms are parallel to each other, lightweight and easy to hold. In addition, the discomfort to the user is minimal to nil.

### BRIEF DESCRIPTION OF THE DRAWINGS

FIG. 1 is an exemplary top perspective of the tool depicting embodiment of scraper attachment and over-molded tips.

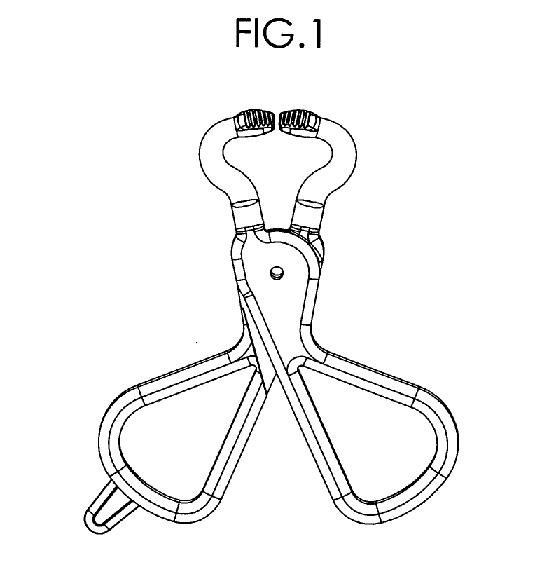

FIG. 2 is the top perspective depicting the embodiment of the tips including soft over-mold.

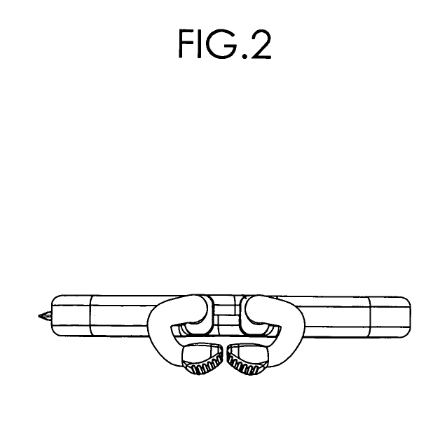

FIG. 3 is the bottom perspective of the tool including the scraper embodiment on left fingerloop.

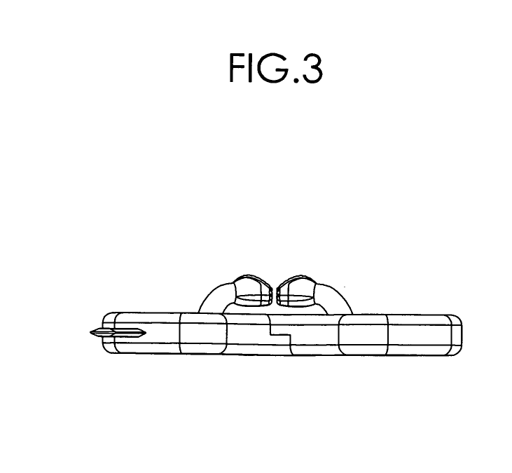

FIG. 4 is the lateral planar perspective depicting the curved tips.

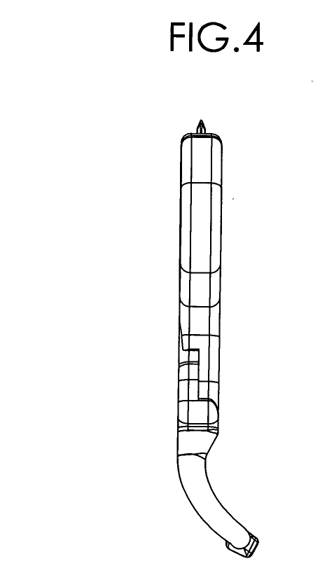

1. A gripping tool used to press skin together and extrude the contents of a clogged pore comprises: 2 opposing arm members pivotally attached by a rivet or screw at a central hinge point with contacting tips at one end of the arm members and handle portions at the opposing end.

2. The handle ends of the arm members are brought together with fingers causing a pivot in the center portion of the arms resulting in the opposing tips pressing firmly together. Said handle members according to claim 1 may be ergonomically curved or designed with fingerloops to aid in opening and closing said tool. The material of the arm members may be comprised of steel, stainless steel, aluminum, polymers, plastic, polyurethane, ceramic or titanium.

3. The contacting tips according to claim 1 come together when force is applied to the opposing handle members. Said tips curve inward from the main planar arm body to disperse the squeezing pressure over a wider surface area and provide greater comfort to the user. The curved opposing tips are molded with a soft material which may be fabricated from rubber, foam, silicone, plastic or polyurethane to provide further comfort to the user. Said tips may be fixed or disposable and applied by methods such as by adhesive or by using a small, mushroom-shaped extension protruding from the surface of the working tip that includes undercuts to hold the overmolded material in place.

4. The handle members are oppositely recessed in the central portion of the tool whereby the arm members can be coupled together. The opposing handle members are pivotally engaged with the use of a screw or rivet inserted through a hole located at the central portion of each of the arm members.

5. One fingerloop according to claim 2 is fabricated with a tab-like extension protruding from the lowermost portion of the fingerloop. Said tab is ‘U’ shaped with the outermost terminal end of the tab having a very small diameter resulting in a sharper edge. This sharper edge allows for removal of blemishes in areas difficult to access using the squeezing portion of the tool. The tab extension is pushed across the surface of the skin with an inward pressure thereby extruding the contents of the affected pore.
